(19) United States
(12) Patent Application Publication
(10) Pub. No.: US 2025/0134858 A1
(43) Pub. Date: May 1, 2025

(54) COMPOSITIONS AND METHODS FOR TREATMENT OF NAFLD AND NASH AND RELATED DYSLIPIDEMIAS

(71) Applicant: EpicentRx, Inc., La Jolla, CA (US)

(72) Inventors: Scott CAROEN, San Francisco, CA (US); Tony REID, San Diego, CA (US); Bryan ORONSKY, Los Altos Hills, CA (US)

(73) Assignee: EpicentRx, Inc., La Jolla, CA (US)

(21) Appl. No.: 18/836,268

(22) PCT Filed: Feb. 10, 2023
(86) PCT No.: PCT/US2023/012834

§ 371 Date: Aug. 6, 2024

Related U.S. Application Data
(60) Provisional application No. 63/308,623, filed on Feb. 10, 2022.

(51) Int. Cl. A61K 31/397; A61K 31/155; A61K 31/355; A61K 31/465; A61K 31/616; A61K 31/7088; A61K 35/14; A61K 35/18; A61K 36/062; A61K 39/395; A61K 45/06; A61P 1/16

(52) U.S. Cl. CPC A61K 31/397; A61K 31/155; A61K 31/355; A61K 31/465; A61K 31/616; A61K 31/7088; A61K 35/14; A61K 35/18; A61K 36/062; A61K 39/3955; A61K 45/06; A61P 1/16

(57) ABSTRACT

Compositions and methods for preventing or treating a disease or a disorder associated with dyslipidemia are provided. This includes prevention or treatment of non-alcoholic fatty liver disease (NAFLD) and/or non-alcoholic steatohepatitis (NASH). The method comprises administering an effective amount of RRx-001, or a pharmaceutically acceptable salt thereof, to the subject in need thereof to reduce at least one factor of the disease or the disorder, such as a hepatic steatosis score, a serum ALT level, a fibrosis marker, a serum AST level, a hepatic level of cholesterol, a hepatic level of triglycerides, insulin resistance, hyperinsulinemia, a serum NLRP3 level, an oxidative or inflammatory stress marker in the subject, and/or a marker associated with low-density lipoprotein (LDL). In some embodiments, the effective amount of RRx-001, or the pharmaceutically acceptable salt thereof, is administered in a pharmaceutical composition that includes a blood product and/or an agent.

### CROSS-REFERENCE TO RELATED APPLICATIONS SECTION

This application claims priority to U.S. Provisional Patent Application Ser. 63/308,623 filed on Feb. 10, 2022, the entire contents of which are hereby incorporated by reference in their entirety.

### FIELD OF THE INVENTION

This disclosure relates to compositions and methods for the treatment of non-alcoholic fatty liver disease (NAFLD), non-alcoholic steatohepatitis (NASH), and related dyslipidemias.

### BACKGROUND

NAFLD is a multisystem disorder that is defined clinicopathologically by the accumulation of lipids in >5% of hepatocytes and the exclusion of secondary causes of fat accumulation. See G. Mazzolini, et al. (2020) Cells 9(11):2458. NAFLD is the most common cause of chronic liver disease in Western countries and it is highly linked to metabolic disease, including obesity, dyslipidemia, hypertension, and diabetes. NAFLD encompasses a wide spectrum of liver damage, which progresses from simple steatosis or non-alcoholic fatty liver (NAFL), a silent disease with a benign prognosis, involving excessive fat storage in liver cells or hepatocytes, to NASH, the latter of which is characterized by hepatic inflammation and hepatocyte injury in steatotic liver tissue, with or without the presence of fibrosis and end-stage liver disease. According to the widespread “two hit” model, which explains that with the occurrence of NAFLD, the first hit involves steatosis, and the second hit includes cellular stresses, such as oxidative stress and inflammation. See Raman, et al. (2006) Can. J. Gastroenterol. 20(5):345-349. Accordingly, NAFLD patients are more susceptible to the toxic effects of alcohol, drugs, and other insults to the liver, which is due to the functional impairment of steatotic hepatocytes. See G. Mazzolini, et al. (2020) Cells 9(11):2458. NAFLD can progress to cirrhosis and hepatocellular carcinoma. No treatments for NAFLD currently exist.

Further, the prevalence of NASH in North America is estimated to be about 24.1%, with the highest prevalence reported in the Middle East and South America. See C. L. Horn, et al. (2022) Hepatol. Commun. 6(1):12-35; and Z. M. Younossi, et al. (2016) Hepatology 64:73-84. This disease currently has no specific therapeutic treatment options. See S. Caroen, et al. (2022) J. Clin. Lipidol. 16(3):e69.

Atherosclerosis, a major risk factor for cardiovascular events, caused by dyslipidemia has both the plaque forming consequence, and an ongoing inflammatory response. Both of these additionally play a part in the formation of NASH. Cholesterol crystals can form in the atherosclerotic lesions, which results in inflammation and can activate the NLRP3 inflammasome. See P. Duewell, et al. (2010) Nature 464(7293):1357-1361. The NLRP3 inflammasome is responsible for the maturation and secretion of the proinflammatory cytokines interleukin-1β (IL-1β) and IL-18 and for the induction of pyroptosis (e.g., a type of inflammatory cell death). Overactivation of the NLRP3 inflammasome can be a driver of various diseases. See L. Urwanisch, et al. (2021) Int. J. Mol. Sci. 22(3):1271. Though the NLRP3 inflammasome plays a crucial role in innate immune-mediated inflammation and contributes to the pathogenesis of multiple autoinflammatory, metabolic, and neurodegenerative diseases, medications targeting the NLRP3 inflammasome are not available for clinical use. See Y. Chen, et al. (2021) Cell Mol. Immunol. 18(6):1425-1436.

The American Heart Association endorses the National Cholesterol Education Program (NCEP) guidelines for the detection of high cholesterol and the guidelines for healthy fasting lipoproteins. It is desirable for an individual's total cholesterol levels to be less than 200 mg/dL, with levels 240 mg/dL and above being considered high. Triglyceride levels of less than 150 mg/dL are considered normal, while triglyceride levels at 200 mg/dL and above are considered high. Low-density lipoprotein (LDL) cholesterol levels at 129 mg/dL or less are considered desirable, with levels at or above 160 mg/dL being considered high. High density lipoprotein (HDL) cholesterol levels at 60 mg/dL and above are considered to be protective against heart disease, with HDL levels less than 40 mg/dL being a major risk factor for heart disease. See Third Report of the National Cholesterol Education Program (NCEP) Expert Panel on Detection, Evaluation and Treatment of High Blood Cholesterol in Adults (Adult Treatment Panel III) Final Report, National Institutes of Health, NIH Publication No. 02-5215 (2002).

Current treatments to reduce elevated triglycerides, total cholesterol, and LDL-cholesterol in patients include small molecule monotherapy and combination therapy. Drugs to achieve these goals include statins, PCSK9 inhibitors, fibric acid derivatives, bile acid sequestrants, nicotinic acid, selective cholesterol absorption inhibitors, red rice yeast, omega 3 fatty acids, fatty acid esters, and adenosine triphosphate-citrate lyase (ACL) inhibitors. Several of these drugs are used in combination therapy, such as ADVICOR® (lovastatin and niacin by Abbott Laboratories); CADUET® (atorvastatin and amlodipine by Pfizer, Inc.); and VYTORIN™ (simvastatin and ezetimibe by Merck and Schering Plough).

However, these therapies have undesirable side effects. For example, statins, which inhibit the HMG-CoA reductase (HMGCR) pathway, are associated with rhabdomyolysis, myopathy, and type II diabetes. Further, niacin, a B vitamin that is used to lower triglycerides and raise HDL levels, is associated with flushing and itching. Thus, novel compositions for the treatment of diseases and disorders associated with high cholesterol are needed that do not have these undesirable side effects.

RRx-001 is a highly selective and potent NLRP3 inhibitor and Nrf2 activator with anti-inflammatory, antioxidative, and antifibrotic properties as a result. See S. Caroen, et al. (2022) J. Clin. Lipidol. 16(3):e69; and Y. Chen, et al. (2021) Cell Mol. Immunol. 18(6):1425-1436. In rat studies, animals fed a high fat diet and treated with RRx-001 had significantly reduced hepatic steatosis scores, reduced cholesterol, and reduced triglycerides as compared to rats fed a high fat diet alone. See Y. Chen, et al. (2021) Cell Mol. Immunol. 18(6):1425-1436. Thus, targeting the NLRP3 inflammasome and activating Nrf2 with RRx-001 contributes to the prevention and treatment of NASH, as well as other disease and disorders associated with dyslipidemia. See S. Caroen, et al. (2022) J. Clin. Lipidol. 16(3):e69.

### SUMMARY

An embodiment of the present invention describes a method for preventing or treating a disease or a disorder, such as NASH or NAFLD, associated with dyslipidemia in a subject. The method comprises administering an effective amount of RRx-001, or a pharmaceutically acceptable salt thereof, to the subject in need thereof. In some embodiments, RRx-001 is represented by a formula:

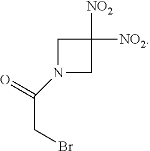 [SMILES: O=C(CBr)N1CC([N+](=O)[O-])([N+](=O)[O-])C1]

In some embodiments, the effective amount of RRx-001, or the pharmaceutically acceptable salt thereof, is a therapeutically effective amount of the RRx-001, or the pharmaceutically acceptable salt thereof. In some embodiments, the subject is a mammal subject. In other embodiments, the mammal subject is a human subject. In some embodiments, the therapeutically effective amount of the RRx-001, or the pharmaceutically acceptable salt thereof, is in a range of from about 0.1 mg to about 500.0 mg. In some embodiments, the therapeutically effective amount of the RRx-001, or the pharmaceutically acceptable salt thereof, is in a range of from about 0.5 mg to about 200.0 mg.

In other embodiments, the mammal subject is a non-human subject. In some embodiments, the therapeutically effective amount of the RRx-001, or the pharmaceutically acceptable salt thereof, is in a range of from about 0.1 mg/kg of the non-human subject to about 100.0 mg/kg of the non-human subject. In some embodiments, the therapeutically effective amount of the RRx-001, or the pharmaceutically acceptable salt thereof, is in a range of from about 1 mg/kg of the non-human subject to about 50.0 mg/kg of the non-human subject.

In some embodiments, the disease, the disorder, or an event associated with the dyslipidemia is selected from the group consisting of: NAFLD, NASH, atherosclerosis, hyperlipidemia, hypercholesterolemia, steatosis, liver fibrosis, coronary heart disease, peripheral vascular disease, chronic kidney disease, stroke, aortic aneurysms, glucose intolerance, and diabetes. In some embodiments, the method for preventing or treating the disease or the disorder associated with the dyslipidemia in the subject reduces at least one factor of the disease or the disorder associated with the dyslipidemia in the subject. In some embodiments, each factor of the at least one factor of the disease or the disorder associated with the dyslipidemia in the subject is selected from the group consisting of: a hepatic steatosis score, a serum alanine transaminase (ALT) level, a serum aspartate transferase (AST) level, a hepatic level of cholesterol, a hepatic level of triglycerides, insulin resistance and/or hyperinsulinemia, a serum NLRP3 level, a fibrosis marker, an oxidative stress marker in the subject, an inflammatory stress marker in the subject such as IL-6, TNF-α, and/or CRP, reflecting systemic inflammation, high levels of reactive oxygen species (ROS) or lipid peroxidation, and a marker associated with LDL.

In some embodiments where the at least one factor comprises the serum NLRP3 level, reduction of the serum NLRP3 level is associated with suppression of NLRP3 inflammasome activation. In some embodiments where the at least one factor comprises the inflammatory or the oxidative stress marker in the subject, the inflammatory or the oxidative stress marker is selected from the group consisting of: Interleukin-1 (IL-1), IL-1 beta (or IL-1p), Interleukin-6 (IL-6), Interleukin-8 (IL-8), Interleukin-18 (IL-18), caspase-1, interferon-γ (IFN-γ), Monocyte chemoattractant protein-1 (MCP-1), Cyclooxygenase-2 (COX-2), C-reactive protein (CRP), isoprostanes (IsoPs), malondialdehyde (MDA), nuclear factor-kappa B (NF-kβ), prostaglandins, and Tumor necrosis factor α (TNFα). In some embodiments where the at least one factor comprises the marker associated with the LDL, the marker associated with the LDL is selected from the group consisting of: serum amyloid A-LDL (SAA-LDL) and α1-antitrypsin-LDL (AT-LDL).

In some embodiments, administering the effective amount of RRx-001, or the pharmaceutically acceptable salt thereof, is performed via a single administration. In other embodiments, administering the effective amount of RRx-001, or the pharmaceutically acceptable salt thereof, is performed via at least two administrations. In some embodiments, administering the effective amount of RRx-001, or the pharmaceutically acceptable salt thereof, is performed at a frequency of at least once per day for a time period, at least once per week for the time period, or at least once per month for the time period.

In some embodiments, administering the effective amount of RRx-001, or the pharmaceutically acceptable salt thereof, occurs via parenteral administration. In other embodiments, administering the effective amount of RRx-001, or the pharmaceutically acceptable salt thereof, occurs via an intravenous administration, intraarterial hepatic administration, an oral administration, intraperitoneal administration, rectal administration, topical administration, vaginal administration, direct intralesional injection, a subcutaneous administration, or an intramuscular administration. In some embodiments, administering the effective amount of RRx-001, or the pharmaceutically acceptable salt thereof, occurs via the intravenous administration.

In some embodiments, the effective amount of RRx-001, or the pharmaceutically acceptable salt thereof, is administered prior to, concurrently with, or subsequent administration of an agent. In some embodiments, the effective amount of RRx-001, or the pharmaceutically acceptable salt thereof, is administered in a pharmaceutical composition comprising an agent. In some embodiments, the effective amount of RRx-001, or the pharmaceutically acceptable salt thereof, is administered in a first pharmaceutical composition and the agent is administered in a second pharmaceutical composition prior to, concurrently with, or subsequent the administration of the first pharmaceutical composition.

In some embodiments, the agent comprises a NASH therapy. The NASH therapy may be an anti-inflammatory agent, an anti-diabetic agent, an anti-fibrotic agent, an anti-steatiotic agent, a cholesterol/lipid modulating agent and/or an anti-diabetic agent. In some embodiments, the NASH therapy is selected from the group consisting of: a statin, a PCSK9 inhibitor, a small interfering RNA, a selective microsomal triglyceride transfer protein inhibitor, an apolipoprotein B antisense oligonucleotide, a monoclonal antibody against angiopoietin-like protein 3 (ANGPTL3), Vitamin E, metformin, aspirin, a peroxisome proliferator-activated receptor (PPAR) agonist, a thyroid hormone receptor beta (THR-β) agonist, insulin, a sulfonylurea, a farnesoid X receptor (FXR) agonist, a glucagon-like peptide 1 (GLP-1) antagonist, a fibric acid derivative, a bile acid sequestrant, nicotinic acid, a selective cholesterol absorption inhibitor, red rice yeast, an omega 3 fatty acid, a fatty acid ester, and an adenosine triphosphate-citrate lyase (ACL) inhibitor.

In some embodiments, the effective amount of RRx-001, or the pharmaceutically acceptable salt thereof, is administered in the pharmaceutical composition comprising a blood product. In some embodiments, the blood product comprises erythrocyte cells. In other embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood. In some embodiments, the effective amount of RRx-001, or a pharmaceutically acceptable salt thereof, is administered in the pharmaceutical composition comprising the agent and/or the blood product.

Another embodiment of the present invention comprises a method for reducing the cholesterol in the subject in need thereof. In some embodiments, the method comprises administering a therapeutically effective dose of the RRx-001, or the pharmaceutically acceptable salt thereof, to the subject in need thereof. In some embodiments, the therapeutically effective dose of the RRx-001, or the pharmaceutically acceptable salt thereof, comprises between about 0.5 mg to about 166 mg for the human subject and comprises between about 3 mg/kg and about 8 mg/kg for the non-human subject. In some embodiments, total cholesterol in the subject is reduced subsequent to the administering of the therapeutically effective dose of the RRx-001, or the pharmaceutically acceptable salt thereof.

Another embodiment of the present invention describes a method for treating the NASH in the subject. In some embodiments, the method comprises administering to the subject in need thereof the therapeutically effective dose of the RRx-001, or the pharmaceutically acceptable salt thereof. In some embodiments, one or more markers for inflammation are reduced subsequent to the administering of the therapeutically effective dose of the RRx-001, or the pharmaceutically acceptable salt thereof.

In some embodiments, the method further comprises mixing the therapeutically effective dose of the RRx-001, or the pharmaceutically acceptable salt thereof, with an aliquot of blood before administering the therapeutically effective dose of the RRx-001, or the pharmaceutically acceptable salt thereof, to the subject in need thereof. In some embodiments, the therapeutically effective dose of the RRx-001, or the pharmaceutically acceptable salt thereof, comprises between about 0.5 mg to about 166 mg for the human subject and comprises between about 3 mg/kg and about 8 mg/kg for the non-human subject. In some embodiments, the therapeutically effective dose of the RRx-001, or the pharmaceutically acceptable salt thereof, for the human subject is between about 50 mg and about 140 mg. In some embodiments, the therapeutically effective dose of the RRx-001, or the pharmaceutically acceptable salt thereof, for the human subject is between about 80 mg and about 130 mg. In some embodiments, the therapeutically effective dose of the RRx-001, or the pharmaceutically acceptable salt thereof, for the human subject is about 166 mg.

In some embodiments, the therapeutically effective dose of the RRx-001, or the pharmaceutically acceptable salt thereof, is administered at the frequency of between about once every day and about once every 60 days. In some embodiments, the therapeutically effective dose of the RRx-001, or the pharmaceutically acceptable salt thereof, is administered at the frequency of between about once every 7 days and about once every 30 days. In some embodiments, the therapeutically effective dose of the RRx-001, or the pharmaceutically acceptable salt thereof, is administered at the frequency of about once every two weeks. In some embodiments, the therapeutically effective dose of the RRx-001, or the pharmaceutically acceptable salt thereof, is administered for a period of between about 4 weeks and about 10 weeks.

A further embodiment of the present invention describes a method for treating atherosclerosis in the subject. In some embodiments, the method comprises administering to the subject in need thereof the therapeutically effective dose of the RRx-001, or the pharmaceutically acceptable salt thereof. In some embodiments, one or more markers for LDL are reduced subsequent to administering the therapeutically effective dose of the RRx-001, or the pharmaceutically acceptable salt thereof, to the subject in need thereof.

In some embodiments, the therapeutically effective dose of the RRx-001, or the pharmaceutically acceptable salt thereof, comprises between about 0.5 mg to about 166 mg for the human subject and comprises between about 3 mg/kg and about 8 mg/kg for the non-human subject. In some embodiments, the therapeutically effective dose of the RRx-001, or the pharmaceutically acceptable salt thereof, for the human subject is between about 50 mg and about 140 mg. In some embodiments, the therapeutically effective dose of the RRx-001, or the pharmaceutically acceptable salt thereof, for the human subject is between about 80 mg and about 130 mg. In some embodiments, the therapeutically effective dose of the RRx-001, or the pharmaceutically acceptable salt thereof, for the human subject is about 166 mg.

In some embodiments, the therapeutically effective dose of the RRx-001, or the pharmaceutically acceptable salt thereof, is administered at the frequency of between about once every day and about once every 60 days. In some embodiments, the therapeutically effective dose of the RRx-001, or the pharmaceutically acceptable salt thereof, is administered at the frequency of between about once every 7 days and about once every 30 days. In some embodiments, the therapeutically effective dose of the RRx-001, or the pharmaceutically acceptable salt thereof, is administered at the frequency of about once every two weeks. In some embodiments, the therapeutically effective dose of the RRx-001, or the pharmaceutically acceptable salt thereof, is administered over a period between about 4 weeks and about 20 weeks.

In some embodiments, the therapeutically effective dose of the RRx-001, or the pharmaceutically acceptable salt thereof, is administered via an intravenous injection mixed with the aliquot of the blood. In some embodiments, the method further comprises using a liposomal preparation.

Another embodiment of the present invention describes a method for reducing at least one of LDL-cholesterol or triglycerides in the subject having the disease that is NAFLD or NASH. In some embodiments, the method comprises administering the therapeutically effective dose of the RRx-001, or the pharmaceutically acceptable salt thereof, to the subject in need thereof. In some embodiments, the therapeutically effective dose of the RRx-001, or the pharmaceutically acceptable salt thereof, is administered in combination with one or more agents, such as NASH therapies. In some embodiments, the method further comprises mixing the therapeutically effective dose of the RRx-001, or the pharmaceutically acceptable salt thereof, with the aliquot of the blood before administering the therapeutically effective dose of the RRx-001, or the pharmaceutically acceptable salt thereof, to the subject in need thereof.

In some embodiments, the therapeutically effective dose of the RRx-001, or the pharmaceutically acceptable salt thereof, comprises between about 0.5 mg to about 166 mg for the human subject and comprises between about 3 mg/kg and about 8 mg/kg for the non-human subject. In some embodiments, the therapeutically effective dose of the RRx-001, or the pharmaceutically acceptable salt thereof, comprises between about 50 mg and about 140 mg for the human subject. In some embodiments, the therapeutically effective dose of the RRx-001, or the pharmaceutically acceptable salt thereof, comprises between about 80 mg and about 130 mg for the human subject. In some embodiments, the therapeutically effective dose of the RRx-001, or the pharmaceutically acceptable salt thereof, comprises about 166 mg for the human subject.

In some embodiments, the therapeutically effective dose of the RRx-001, or the pharmaceutically acceptable salt thereof, is administered at the frequency of between about once every day and about once every 60 days. In some embodiments, the therapeutically effective dose of the RRx-001, or the pharmaceutically acceptable salt thereof, is administered at the frequency of between about once every 7 days and about once every 30 days. In some embodiments, the therapeutically effective dose of the RRx-001, or the pharmaceutically acceptable salt thereof, is administered at the frequency of about once every two weeks. In some embodiments, the therapeutically effective dose of the RRx-001, or the pharmaceutically acceptable salt thereof, is administered at the frequency between about 4 weeks and about 10 weeks.

Another embodiment of the present invention describes the pharmaceutical composition that treats the disease or the disorder associated with the dyslipidemia in the subject. The pharmaceutical composition comprises the effective amount of RRx-001, or the pharmaceutically acceptable salt thereof. In some embodiments, the pharmaceutical composition further comprises the blood product and/or the agents, such as the NASH therapies. In some embodiments, the pharmaceutical composition is formulated as an aerosol formulation.

These and other aspects will become apparent from the following detailed description by reference to the figures.

### BRIEF DESCRIPTION OF THE DRAWINGS

FIG. 1A shows a bar graph of a relative amount of LDL in rats with a normal diet (ND), a high fat diet (HFD), and a high fat diet and treated with RRx-001 (HFD-RRx-001), according to at least some embodiments disclosed herein.

FIG. 1B shows a bar graph of a relative amount of high density lipoprotein (HDL) in rats with a ND, a HFD, and a HFD-RRx-001, according to at least some embodiments disclosed herein.

FIG. 1C shows a bar graph of a relative amount of triglyceride (TG) in rats with a ND, a HFD, and a HFD-RRx-001, according to at least some embodiments disclosed herein.

FIG. 1D shows a bar graph of a relative amount of total cholesterol (TC) in rats with a ND, a HFD, and a HFD-RRx-001, according to at least some embodiments disclosed herein.

FIG. 2A is an image of oil red O staining of aortic tissue of a ND rat, according to at least some embodiments disclosed herein.

FIG. 2B is an image of oil red O staining of aortic tissue of a HFD rat, according to at least some embodiments disclosed herein.

FIG. 2C is an image of oil red O staining of aortic tissue of a HFD rat that is treated with RRx-001, according to at least some embodiments disclosed herein.

FIG. 3A shows a bar graph of a relative amount of hepatic TG in rats with a ND, a HFD, and a HFD-RRx-001, according to at least some embodiments disclosed herein.

FIG. 3B shows a bar graph of a relative amount of hepatic TC in rats with a ND, a HFD, and a HFD-RRx-001, according to at least some embodiments disclosed herein.

FIG. 4A is an image of oil red O staining of tissue of a ND rat, according to at least some embodiments disclosed herein.

FIG. 4B is an image of oil red O staining of tissue of a HFD rat, according to at least some embodiments disclosed herein.

FIG. 4C is an image of oil red O staining of tissue of a HFD rat that is treated with RRx-001, according to at least some embodiments disclosed herein.

FIG. 4D is an image of hemotoxylin and eosin (H&E) staining of tissue of a ND rat, according to at least some embodiments disclosed herein.

FIG. 4E is an image of H&E staining of tissue of a HFD rat, according to at least some embodiments disclosed herein.

FIG. 4F is an image of H&E staining of tissue of a HDF rat that is treated with RRx-001, according to at least some embodiments disclosed herein.

FIG. 5A shows a bar graph of a relative amount of NLR family pyrin domain containing 3 (NLRP3) in rats with a ND, a HFD, and a HFD-RRx-001, according to at least some embodiments disclosed herein.

FIG. 5B shows a bar graph of a relative amount of IL-1β in rats with a ND, a HFD, and a HFD-RRx-001, according to at least some embodiments disclosed herein.

FIG. 5C shows a bar graph of a relative amount of caspase-1 in rats with a ND, a HFD, and a HFD-RRx-001, according to at least some embodiments disclosed herein.

### DETAILED DESCRIPTION

Unless specifically defined otherwise all technical and scientific terms used herein shall be taken to have the same meaning as commonly understood by one of ordinary skill in the art.

### Definitions

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The term “comprise” or variations such as “comprises” or “comprising” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

“Treatment,” “treating” or similar phrases refer to obtaining beneficial or desired results, such as clinical results, for a subject, suffering from the disease, the disorder, or an event associated with the dyslipidemia (e.g., NAFLD, NASH, atherosclerosis, hyperlipidemia, hypercholesterolemia, steatosis, liver fibrosis, glucose intolerance, coronary heart disease, peripheral vascular disease, chronic kidney disease, stroke, aortic aneurysms, or diabetes). As used herein, the term “treating” includes any effect, e.g., lessening, reducing, modulating, ameliorating or eliminating, that results in the improvement of the condition, the disease, the disorder, and the like, or ameliorating a symptom thereof. Beneficial or desired results include any one or more of: alleviating one or more symptoms of the disease or the disorder, diminishing the extent of the disease or the disorder, delaying or slowing progression of the disease or the disorder, and improving quality of life.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “subject” refers to an organism to be treated by the methods of the present invention. Such organisms are preferably mammals (e.g., murines, simians, equines, bovines, porcines, canines, felines, and the like), and more preferably humans.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, intraarterial hepatic, subcuticular, intraarticulare, subcapsular, subarachnoid, intraspinal, and intrasternal injection and infusion.

As used herein, the term “pharmaceutical composition” refers to the combination of an active agent with an excipient or a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. See Martin, Remington's Pharmaceutical Sciences, 15th Ed., Mack Publ. Co., Easton, PA [1975].

As used herein, the term “pharmaceutically acceptable salt” refers to any circular salt (e.g., acid or base) of a compound of the present invention suitable for pharmaceutical administration which, upon administration to a subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW4+, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

The term “excipient” as used herein means an inert or inactive substance that may be used in the production of a drug or pharmaceutical, such as an inhalable formulation containing a compound described herein as an active ingredient. Various substances may be embraced by the term excipient, including without limitation any substance used as a surfactant, emulsifying agent, anti-oxidants, pH adjusting agent, preservative, lubricant, or solvent for pulmonary administrable solutions. Discussions related to excipients may be found, e.g., in Remington: The Science and Practice of Pharmacy, Lippincott Williams & Wilkins, 21^{st} ed. (2005), and Pharmaceutics: The science of dosage form design, M. E. Aulton, (1988).

The terms “ABDNAZ” and “RRx-001” are used interchangeably and refer to the compound having the following structure:

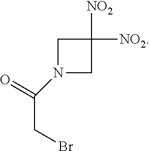 [SMILES: O=C(CBr)N1CC([N+](=O)[O-])([N+](=O)[O-])C1]

RRx-001 is associated with the chemical name 2-bromo-1-(3,3-dinitroazetidin-1-yl)ethan-1-one and is a small cyclic nitro compound that is a minimally toxic anticancer agent in Phase 3 clinical trial for the treatment of cancer. RRx-001 has been shown to demonstrate anti-cancer activity through epigenetic, antiangiogenic, antioxidant, apoptotic and nitric oxide sub-mechanisms. See Pedro Cabrales (2019) Transl. Oncol. 12(4):626-632; and B. Oronsky, et al. (2017) Med. Oncol. 34(6):103. Methods of synthesizing ABDNAZ have been described, such as in U.S. Pat. Nos. 7,507,842 and 8,471,041.

Further, RRx-001 is an electrophilic stress regulator with anti-oxidative/anti-inflammatory, vasodilatory, and cardioprotective properties. These effects are mediated by Nrf2 activation and NLRP3 inhibition, as well as nitric oxide generation under hypoxia. NLRP3 is expressed in immune cells, especially in dendritic cells and macrophages and acts as a constituent of the inflammasome. See Ghafouri-Fard (2022) Front. Immunol. 13:926895. NLRP3 acts as a pattern recognition receptor identifying pathogen-associated molecular patterns and also recognizes damage-associated molecular patterns. See Ghafouri-Fard (2022) Front. Immunol. 13:926895. Triggering of NLRP3 inflammasome by molecules ATP released from injured cells results in the activation of the inflammatory cytokines IL-1β and IL-18. Abnormal activation of NLRP3 inflammasome stimulates inflammatory or metabolic diseases. See Ghafouri-Fard (2022) Front. Immunol. 13:926895. As such, NLRP3 is a target for decreasing activity of NLRP3 inflammasome. RRx-001 ameliorates inflammatory diseases by acting as a potent covalent NLRP3 inhibitor. See Y. Chen, et al. (2021) Cell Mol. Immunol. 18(6):1425-2436; and M. Ma, et al. (2021) Front. Immunol. 12:718779. Thus, RRx-001 is thought to be an inhibitor of not only cancer, but also inflammatory conditions.

In fact, intraperitoneal administration of RRx-001 has been shown to improve high-fat diet-induced NASH in rats, resulting in reduced hepatic steatosis scores, serum ALT and AST levels, and hepatic levels of cholesterol and triglycerides. These findings were correlated with the suppression of the NLRP3 inflammasome activation by RRx-001, as evidenced by decreased levels of NLRP3, caspase-1, and IL-1β levels. See S. Caroen, et al. (2022) J. Clin. Lipidol. 16(3):e69.

RRx-001 reduces cholesterol levels in serum or hypercholesterolemia, which is associated with fatty liver and atherosclerosis. Traditional standards to reduce cellular levels of cholesterol have undesirable side effects in the subject. For example, statins are associated with rhabdomyolysis, myopathy, and type II diabetes. Distinctly, RRx-001 does not cause muscle breakdown or hyperglycemia and is likely to lower cholesterol levels through a different pathway. Further, niacin, a B vitamin that is used to lower triglycerides and raise HDL levels, results in flushing and itching. RRx-001 is not associated with these side effects. Additionally, unlike fibric acid derivatives fenofibrate and gemfibrozil, RRx-001 has been shown to dramatically decrease hepatic fat and inflammatory cytokines, as well as to improve the LDL phenotype. Since the presence of hepatic fat content in obese subjects is predictive of both NAFLD and NASH, reduction of hepatic fat by RRx-001 is significant in the treatment of NAFLD and NASH.

### Methods of Treatment

In some embodiments, the method for preventing or treating the disease or the disorder associated with the dyslipidemia in the subject comprises administering an effective amount of a compound of Formula A:

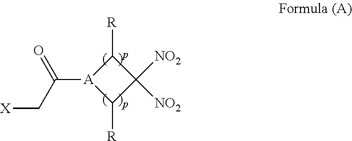

or a pharmaceutically acceptable salt thereof, wherein:
A is —N or —C(H),
R is independently hydrogen or methyl,
p is independently 1 or 2, and
X represents a halogen.

In some embodiments, the effective amount of the compound of Formula (A), or the pharmaceutically acceptable salt thereof, is a therapeutically effective amount of the compound of Formula (A), or the pharmaceutically acceptable salt thereof.

In some examples, the method for treating the disease or the disorder and the related dyslipidemia in the subject comprises administering an effective amount of RRx-001, or a pharmaceutically acceptable salt thereof, to the subject in need thereof. In some embodiments, the effective amount of RRx-001, or the pharmaceutically acceptable salt thereof, is a therapeutically effective amount of the RRx-001, or the pharmaceutically acceptable salt thereof.

In some embodiments, the disease, the disorder, or the event associated with the dyslipidemia is selected from the group consisting of: NAFLD, NASH, atherosclerosis, hyperlipidemia, hypercholesterolemia, steatosis, liver fibrosis, coronary heart disease, glucose intolerance, peripheral vascular disease, chronic kidney disease, stroke, aortic aneurysms, and diabetes. In some embodiments, the subject is a mammal subject. In some embodiments, the mammal subject is a human subject. In other embodiments, the mammal subject is a non-human subject.

In some embodiments, administering the effective amount of RRx-001, or the pharmaceutically acceptable salt thereof, to the subject in need thereof reduces at least one factor of the disease or the disorder associated with the dyslipidemia. In some embodiments, each factor of the at least one factor of the disease or the disorder associated with the dyslipidemia in the subject is selected from the group consisting of: a hepatic steatosis score, a serum ALT level, a serum AST level, a hepatic level of cholesterol, a hepatic level of triglycerides, insulin resistance and/or hyperinsulinemia, an NLRP3 level, an inflammatory stress marker in the subject such as IL-6, TNF-α, and CRP, reflecting systemic inflammation, high levels of ROS or lipid peroxidation, a fibrosis marker, and a marker associated with LDL.

In some embodiments, the at least one factor of the disease or the disorder associated with the dyslipidemia is a hepatic steatosis score or index (HSI). The HSI index determines the likelihood of an NAFLD diagnosis based on a gender of the subject, AST, ALT, and body mass index (BMI). See J. Lee, et al. (2010) Dig. Liver Dis. 42(7):503-508. Based on the results of multiple logistic regression analysis, the HSI equation is: HSI=8×ALT/AST+BMI(+2 if type 2 diabetes yes, +2 if female). The BMI is calculated using the following: body weight (kg)/height squared (m²). See J. Lee, et al. (2010) Dig. Liver Dis. 42(7):503-508. Once the HIS index is calculated, it is interpreted by a comparison to the two cut-off points: (1) HSI values below 30 indicate that NAFLD can be ruled out and (2) HSI values of 36 and above indicate that an NAFLD positive diagnosis is likely. See J. Lee, et al. (2010) Dig. Liver Dis. 42(7):503-508.

In some embodiments, the at least one factor of the disease or the disorder associated with the dyslipidemia is the serum ALT level. The serum ALT level is a marker of NAFLD, predicts incident type 2 diabetes mellitus, and is associated with endothelial dysfunction and carotid atherosclerosis. See R. Schindhelm, et al. (2007) Atherosclerosis 191(2):P391-396. The normal range for the serum ALT level is between about 7 units/liter (U/L) to about 56 U/L.

In other embodiments, the at least one factor of the disease or the disorder associated with the dyslipidemia is the serum AST level. The normal range for the serum AST level is between about 8 U/L to about 33 U/L. A serum AST level above this range may be a sign of chronic hepatitis, damage from alcohol, cholestasis, liver cancer, or cirrhosis.

In some embodiments, the at least one factor of the disease or the disorder associated with the dyslipidemia is the hepatic level of cholesterol. In other embodiments, the at least one factor of the disease or the disorder associated with dyslipidemia is a hepatic level of triglycerides. In some embodiments, the at least one factor of the disease or the disorder associated with dyslipidemia is a serum NLRP3 level. Reduction of the serum NLRP3 level is associated with suppression of NLRP3 inflammasome activation.

In some embodiments, the at least one factor of the disease or the disorder associated with the dyslipidemia is the oxidative or the inflammatory stress marker in the subject. In some embodiments, the oxidative or inflammatory stress marker is selected from the group consisting of: IL-1, IL-1β, IL-6, IL-8, IL-18, caspase-1, IFN-γ, MCP-1, COX-2, CRP, NF-kβ, IsoPs, MDA, prostaglandins, and TNFα.

In other embodiments, the at least one factor of the disease or the disorder associated with the dyslipidemia is the marker associated with the LDL. In some embodiments, the marker associated with the LDL is selected from the group consisting of: SAA-LDL and AT-LDL. The SAA-LDL complex is formed from the oxidative interaction between SAA and LDL lipoproteins. The AT-LDL complex is formed by the binding between oxidized α1-antitrypsin and LDL in the intimal layer of the arterial wall. It has been suggested that circulating AT-LDL may reflect the activity of foam cells in atherosclerotic lesions. See K. Kotani, et al. (2009) Atherosclerosis 204(2):526-531.

In some embodiments, the method comprises administering one or more additional therapeutic or diagnostic agents (such as the NASH therapies) to the subject prior to, concurrently with, or subsequent administering the effective amount of the RRx-001, or the pharmaceutically acceptable salt thereof to the subject. In some embodiments, the NASH therapy may be an anti-inflammatory agent, an anti-diabetic agent, an anti-fibrotic agent, an anti-steatiotic agent, a cholesterol/lipid modulating agent and/or an anti-diabetic agent. In some embodiments, the NASH therapy is selected from the group consisting of: a statin (e.g., atorvastatin, cerivastatin, fluvastatin, mevastatin, pitavastatin, lovastatin, provastatin, rosuvastatin, and simvastatin, among others), a PCSK9 inhibitor (e.g., alirocumab, evolocumab, inclisiran, Praluent, and Repatha, among others), a fibric acid derivative (e.g., Trillipix, fenofibric acid, fenofibrate, clofibrate, tricor, lopid, gemfibrozil, lipofen, and antara, among others), a bile acid sequestrant (e.g., colesevelam, cholestyramine, and colestipol, among others), nicotinic acid, a selective cholesterol absorption inhibitor (e.g., ezetimibe), red rice yeast, an omega 3 fatty acid, a fatty acid ester, an ACL inhibitor (e.g., bempedoic acid), metformin, aspirin, a PPAR agonist, a THR-β agonist, a GLP1-receptor agonist, a sulfonylurea, vitamin E, a small interfering RNA, a selective microsomal triglyceride transfer protein inhibitor, an apolipoprotein B antisense oligonucleotide, a monoclonal antibody against angiopoietin-like protein 3 (ANGPTL3), insulin, and a farnesoid X receptor (FXR) agonist.

### Methods for Administering RRx-001

The therapeutically effective amount of the RRx-001, or the pharmaceutically acceptable salt thereof, may be administered to the subject through a variety of methods. In some embodiments, the therapeutically effective amount of the RRx-001, or the pharmaceutically acceptable salt thereof, may be administered through a vehicle, such as dimethyl sulfoxide (DMSO).

In an exemplary embodiment, a subject's blood is withdrawn and extracorporeally combined with the therapeutically effective amount of the RRx-001, or the pharmaceutically acceptable salt thereof. The composition, which includes the therapeutically effective amount of the RRx-001, or the pharmaceutically acceptable salt thereof, and the blood product, is then re-introduced back to the subject.

### Pharmaceutical Composition

The present disclosure provides pharmaceutical compositions. As a general matter, the pharmaceutical composition contains at least one active agent and a pharmaceutically acceptable carrier. The pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders can contain, in addition to a compound of this invention, excipients such as lactose, talc, silicic acid, aluminum hydroxide, calcium silicates and polyamide powder, or mixtures of these substances. Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel. Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

In some embodiments, the pharmaceutical composition is configured as an inhalable formulation that comprises an effective amount of RRx-001, or a pharmaceutically acceptable salt thereof, and at least one pharmaceutically acceptable carrier or excipient. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation or mouth inhalation, or both.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. Nos. 6,932,962 B1 and 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof, a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. Nos. 6,932,962 B1 and 8,367,734 B1.

In some embodiments, the aerosol formulation is propellant-free and comprises the effective amount of RRx-001, or the pharmaceutically acceptable salt thereof, and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof, such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof. Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation with the propellant may be packed in and/or dispensed by a metered dose inhaler (MDI). In some embodiments, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen.

When the compounds of the present invention are administered as pharmaceuticals to subjects, they can be given per se or as a pharmaceutical composition containing, for example, 0.1 to 99% (more preferably, 10 to 30%) of active ingredient in combination with a pharmaceutically acceptable carrier.

Combination with Blood Product and/or Another Agent

In certain embodiments, the pharmaceutical compositions comprise pharmaceutical compositions for treating the disease or the disorder associated with the dyslipidemia in the subject, where the pharmaceutical composition comprises (1) an effective amount of RRx-001, or a pharmaceutically acceptable salt thereof and (2) a blood product and/or (3) one or more additional therapeutic or diagnostic agents (such as the NASH therapies).

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood.

In some embodiments, the NASH therapy may be an anti-inflammatory agent, an anti-diabetic agent, an anti-fibrotic agent, an anti-steatiotic agent, a cholesterol/lipid modulating agent and/or an anti-diabetic agent. In some embodiments, the NASH therapy is selected from the group consisting of: a statin, a PCSK9 inhibitor, a small interfering RNA, a selective microsomal triglyceride transfer protein inhibitor, an apolipoprotein B antisense oligonucleotide, a monoclonal ANGPTL3, Vitamin E, metformin, aspirin, a PPAR agonist, a THR-β agonist, insulin, a sulfonylurea, a FXR agonist, a GLP-1 antagonist, a fibric acid derivative, a bile acid sequestrant, nicotinic acid, a selective cholesterol absorption inhibitor, red rice yeast, an omega 3 fatty acid, a fatty acid ester, a sulfonylurea, and an ACL inhibitor. The drawbacks associated with statin treatment, discussed above, e.g., risk of type 2 diabetes and/or rhabdomyolysis and myopathy, may be attenuated when a statin is used in conjunction with an anti-inflammatory like RRx-001.

In some embodiments, the therapeutic or diagnostic agent in the pharmaceutical composition is subject to a reduced incidence of drug-drug interaction as compared to direct administration of the same therapeutic or diagnostic agent at the same dose without being mixed with the blood product prior to administration. In certain embodiments, the reduced incidence of drug-drug interaction permits the use of a second therapeutic or diagnostic agent that would have otherwise been contraindicated.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, as described herein, “attenuating interactions” of drugs refers to actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of either or both therapeutic or diagnostic agents or both pharmaceutical compositions. In some embodiments, the attenuated interactions can impact the transport or the distribution of the therapeutic or diagnostic agents or the pharmaceutical compositions.

A therapeutic agent may cause significant side effects or toxicity when administered to the subject at a therapeutically effective dose, without the blood mix of the present invention. Methods of the present invention can provide improved efficacy and/or reduced toxicity when a therapeutic agent is administered to a subject by a blood-based delivery. Therefore, with the present invention, the therapeutic agent can be administered to a subject in a blood mix at a higher, more therapeutically effective dose, but still has comparable or reduced toxicity compared to the situation where the therapeutic agent is administered to the subject without the blood mix.

Accordingly, in certain embodiments, the subject has reduced incidence and/or severity of side effects compared to subjects receiving a direct administration of the same therapeutic agent at the same dose without being mixed with the blood product prior to administration. In certain embodiments, the subject has reduced side effects compared to subjects receiving a direct administration of the same therapeutic agent at the same dose without being mixed with the blood product prior to administration. In certain embodiments, the dose of the therapeutic agent in the pharmaceutical composition is at least about 10% to about 300% more than the dose recommended for a direct administration of the same therapeutic agent without being mixed with the blood product prior to administration. In certain embodiments, the dose of the therapeutic agent in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same therapeutic agent without being mixed with the blood product prior to administration.

In certain embodiments, the therapeutic agent has a longer circulating half-life in the subject compared to direct administration of the same therapeutic agent at the same dose without being mixed with the blood product prior to administration. In certain embodiments, the circulating half-life of the therapeutic agent is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190°/a, 200%, 250%, 300%, 350%, 400%, 450%, 500%, 600%, 700%, 800%, 900%, 1000%, or more, longer than the circulating half-life of the same therapeutic agent at the same dose without being mixed with the blood product before administration.

In some embodiments, the side effects/toxicities include, but are not limited to, pulmonary toxicity (e.g., interstitial infiltrates, noncardiogenic pulmonary edema, and/or pulmonary hemorrhage), cardiovascular toxicity (e.g., hypertension), vascular toxicity (e.g., arteriothromboembolic, venous, and/or pericardial effusions), hepatotoxicity (e.g., fatty liver, veno-occlusive disease, pseudocirrhosis, and/or bilary stricture), pancreas toxicity, pancreatitis toxicity, gastrointestinal toxicity (e.g., enteritis, neutropenic colitis, pneumatosis or perforation, megacolon), genitourinary toxicity (e.g., hemorrhagic cystitis and/or neurogenic bladder), peritoneum, mesentery, or soft tissues toxicity (e.g., ascites), and neurologic toxicity (e.g., peripheral neuropathy, and/or central nervous system), ocular toxicity, and ototoxicity (e.g. hearing loss).

In certain embodiments, the toxicity is caused by use of statins. The toxicity can be detected and measured by any suitable method. The toxicity can increase the risk of type 2 diabetes, rhabdomyolysis and myopathy. For example, rhabdomyolysis and myopathy can cause severe muscle aches or weakness and be measured by a blood test measuring levels of the enzyme creatinine kinase.

### Amount of RRx-001

The therapeutically effective amount of the RRx-001, or the pharmaceutically acceptable salt thereof, may be administered to the subject in various dosages at various frequencies and over various periods of time. Each of the dosage, frequency of the dosage, and the periods of time to which the dosage is administered may be adjusted based on factors, including, but not limited, to the medical condition of the subject, the desired outcome, and the one or more (if any) agents that are administered concurrently with the therapeutically effective amount of the RRx-001, or the pharmaceutically acceptable salt thereof.

In various embodiments, the dosage may be measured in units of a mass of the RRx-001 or the pharmaceutically acceptable salt thereof, a volume of the RRx-001 or the pharmaceutically acceptable salt thereof, a mass of the RRx-001 or the pharmaceutically acceptable salt thereof to a mass of the subject, a mass of the RRx-001 or the pharmaceutically acceptable salt thereof to a volume of the subject, a volume of the RRx-001 or the pharmaceutically acceptable salt thereof to a volume of the subject, a mass of the RRx-001 or the pharmaceutically acceptable salt thereof to a surface area of the subject, a volume of the RRx-001 or the pharmaceutically acceptable salt thereof to a surface area of the subject, or the like.

In some embodiments, the therapeutically effective amount of the RRx-001, or the pharmaceutically acceptable salt thereof, may be administered in one or more doses in a range of about 0.1 mg and about 500.0 mg. In some embodiments, the therapeutically effective amount of the RRx-001, or the pharmaceutically acceptable salt thereof, may be administered in one or more doses in a range of about 0.1 mg and about 200.0 mg. In some embodiments, the therapeutically effective amount of the RRx-001, or the pharmaceutically acceptable salt thereof, may be administered in one or more doses in a range of about 0.1 mg and about 0.5 mg, about 0.5 mg and about 1.0 mg, about 1.0 mg and about 1.5 mg, about 1.5 mg and about 2.0 mg, about 2.0 mg and about 2.5 mg, about 2.5 mg and about 3.0 mg, about 3.0 mg and about 3.5 mg, about 3.5 mg and about 4.0 mg, about 4.0 mg and about 4.5 mg, about 4.5 mg and about 5.0 mg, about 5.0 mg and about 6.0 mg, about 6.0 mg and about 7.0 mg, about 7.0 mg and about 8.0 mg, about 8.0 mg and about 9.0 mg, about 9.0 mg and about 10.0 mg, about 10.0 mg and about 15.0 mg, about 15.0 mg and about 20.0 mg, about 20.0 mg and about 25.0 mg, about 25.0 mg and about 30.0 mg, about 30.0 mg and about 35.0 mg, about 35.0 mg and about 40.0 mg, about 40.0 mg and about 45.0 mg, about 45.0 mg and about 50.0 mg, about 50.0 mg and about 55.0 mg, about 55.0 mg and about 60.0 mg, about 60.0 mg and about 65.0 mg, about 65.0 mg and about 70.0 mg, about 70.0 mg and about 75.0 mg, about 75.0 mg and about 80.0 mg, about 80.0 mg and about 85.0 mg, about 85.0 mg and about 90.0 mg, about 90.0 mg and about 95.0 mg, about 95.0 mg and about 100.0 mg, about 100.0 mg and about 110.0 mg, about 110.0 mg and about 120.0 mg, about 120.0 mg and about 130.0 mg, about 130.0 mg and about 140.0 mg, about 140.0 mg and about 150.0 mg, about 150.0 mg and about 160.0 mg, about 160.0 mg and about 170.0 mg, about 170.0 mg and about 180.0 mg, about 180.0 mg and about 190.0 mg, or about 190.0 mg and about 200.0 mg. In other embodiments, the therapeutically effective amount of the RRx-001, or the pharmaceutically acceptable salt thereof, may be administered in one or more doses in a range of about 100.0 mg and about 120.0 mg, about 120.0 mg and about 140.0 mg, about 140.0 mg and about 160.0 mg, about 160.0 mg and about 180.0 mg, about 200.0 mg and about 220.0 mg, about 220.0 mg and about 240.0 mg, about 240.0 mg and about 260.0 mg, about 260.0 mg and about 280.0 mg, about 280.0 mg and about 300.0 mg, about 300.0 mg and about 320.0 mg, about 320.0 mg and about 340.0 mg, about 340.0 mg and about 360.0 mg, about 360.0 mg and about 380.0 mg, about 380.0 mg and about 400.0 mg, about 400.0 mg and about 420.0 mg, about 420.0 mg and about 440.0 mg, about 440.0 mg and about 460.0 mg, about 460.0 mg and about 480.0 mg, or about 480.0 mg and about 500.0 mg.

In some embodiments, where the subject is a human subject, the therapeutically effective amount of the RRx-001, or the pharmaceutically acceptable salt thereof is in a range of from about 0.1 mg to about 500.0 mg, and more preferably, in the range from about 0.5 mg to about 200.0 mg. In other embodiments, where the subject is a non-human subject, the therapeutically effective amount of the RRx-001, or the pharmaceutically acceptable salt thereof is in a range of from about 0.1 mg/kg of the non-human subject to about 100.0 mg/kg of the non-human subject, and more preferably, in the range from about 1 mg/kg of the non-human subject to about 50.0 mg/kg of the non-human subject.

### Frequency of Administration

In some embodiments, the therapeutically effective amount of the RRx-001, or the pharmaceutically acceptable salt thereof may be administered via a single administration. In some embodiments, the therapeutically effective amount of the RRx-001, or the pharmaceutically acceptable salt thereof, may be administered via at least two administrations. In other embodiments, each administration dosage of the therapeutically effective amount of the RRx-001, or the pharmaceutically acceptable salt thereof may be administered at various intervals over a time period until the end of a dosing period. The various intervals are referred to herein as a “frequency.”

In some embodiments, each dose of the therapeutically effective amount of the RRx-001, or the pharmaceutically acceptable salt thereof, is administered at a frequency of about 1 dose per 24 hours. In another embodiment, each dose of the therapeutically effective amount of the RRx-001, or the pharmaceutically acceptable salt thereof, is administered at a frequency of about 1 dose per 48 hours. In some embodiments, each dose of the therapeutically effective amount of the RRx-001, or the pharmaceutically acceptable salt thereof, is administered at a frequency of about 3 doses per week. In another embodiment, each dose of the therapeutically effective amount of the RRx-001, or the pharmaceutically acceptable salt thereof, is administered at a frequency of about 2 doses per week. In some embodiments, each dose of the therapeutically effective amount of the RRx-001, or the pharmaceutically acceptable salt thereof, is administered at a frequency of about 1 dose per week. In other embodiments, each dose of the therapeutically effective amount of the RRx-001, or the pharmaceutically acceptable salt thereof, is administered at a frequency of about 2 doses per month. In some embodiments, each dose of the therapeutically effective amount of the RRx-001, or the pharmaceutically acceptable salt thereof, is administered at a frequency of about 1 dose per month. In other embodiments, each dose of the therapeutically effective amount of the RRx-001, or the pharmaceutically acceptable salt thereof, is administered at a frequency of about 1 dose per 2 months. In some embodiments, each dose of the therapeutically effective amount of the RRx-001, or the pharmaceutically acceptable salt thereof, is administered at a frequency of about 1 dose per 3 months.

In other embodiments, each dose of the therapeutically effective amount of the RRx-001, or the pharmaceutically acceptable salt thereof, is administered at a frequency of between about 1 dose per day and about 1 dose per week until the end of a dosing period. In some embodiments, each dose of the therapeutically effective amount of the RRx-001, or the pharmaceutically acceptable salt thereof, is administered at a frequency of between about 1 dose per day and about 2 doses per week until the end of a dosing period. In other embodiments, each dose of the therapeutically effective amount of the RRx-001, or the pharmaceutically acceptable salt thereof, is administered at a frequency of between about 1 dose per week and about 1 dose per month until the end of a dosing period. In other embodiments, each dose of the therapeutically effective amount of the RRx-001, or the pharmaceutically acceptable salt thereof, is administered at a frequency of between about 1 dose per month and about 1 dose per 3 months until the end of a dosing period.

In some embodiments, the therapeutically effective amount of the RRx-001, or the pharmaceutically acceptable salt thereof, may be administered at a frequency of once per day, once per week, once per month, etc. during a time period.

### EXAMPLE

The following Example is set forth to enable this disclosure to be more fully understood. It should be understood that this example is for illustrative purposes only and is not to be construed as limiting this disclosure in any manner.

Sprague-Dawley rats (6-weeks-old male weighing about 180 grams) were randomly divided into three groups. The first group of the rats were subjected to a normal diet (ND) for a time period of 12 weeks. The second and the third group of the rats were subjected to a high-fat atherogenic diet (HFD, 40 kcal % fat, 1.25% cholesterol, 0.5% sodium cholate) for the time period of 12 weeks. The third HFD group of the rats were treated with about 5 mg/kg of RRx-001 every other week for the last 8 weeks before characterization (“HFD-RRx-001”). Several markers of inflammation and steatosis were evaluated.

In a trial that tested for markers related to NASH, every other week administrations of RRx-001 reduced hepatic steatosis scores, serum ALT and AST levels, and hepatic levels of cholesterol and triglycerides in rats fed the HFD. These results correlated with the suppression of the NLRP3 inflammasome activation by RRx-001 as evidenced by decreased levels of serum NLRP3, caspase-1, and IL-1β levels on ELISA.

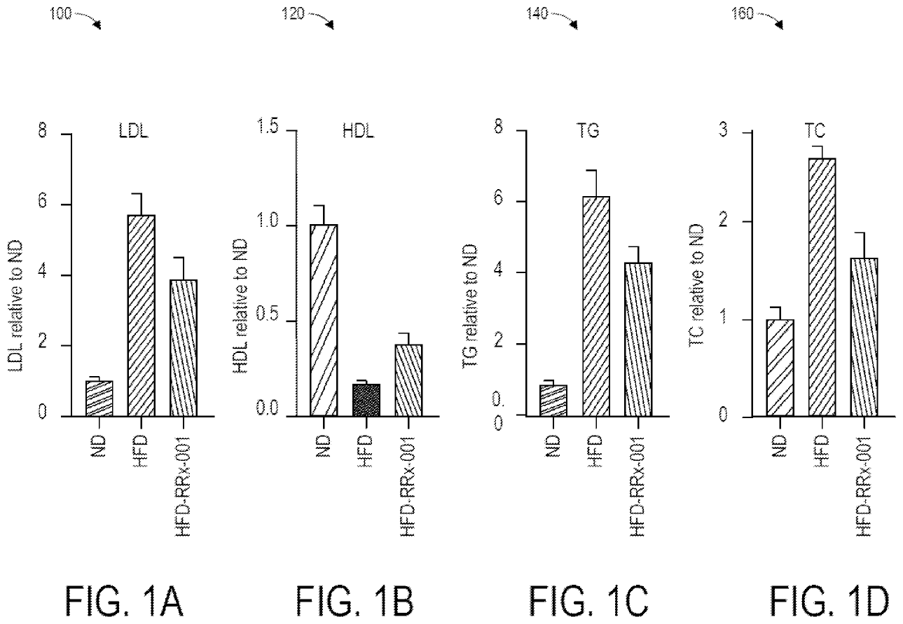

FIG. 1A depicts a bar graph 100 of a relative amount of LDL in the first group of the rats that were fed the ND, the second group of the rats that were fed the HFD, and the third group of the rats that were fed the HFD and were treated with RRx-001 (HFD-RRx-001). The LDL performs a role of delivering cholesterol to peripheral tissues where they may be stored as a source of energy, and as such, may be associated with a building up of fat in an individual. The LDL is widely recognized as a contributor to atherosclerosis whereby plaque is deposited in the lumen of arteries and can lead to heart attack, stroke, and peripheral vascular disease.

As shown in FIG. 1A, the ND rats (e.g., the first group) were measured to have an amount of the LDL that was normalized to 1.0. The LDL of the HFD rats (e.g., the second group) was measured to be approximately 6 times the LDL amount of the ND rats (e.g., the first group). The LDL of the HFD-RRx-001 rats (e.g., the third group) was measured to be approximately 4 times the LDL amount of the ND rats (e.g., the first group).

FIG. 1B depicts a bar graph 120 of a relative amount of HDL in the first group of the rats that were fed the ND, the second group of the rats that were fed the HFD, and the third group of the rats that were fed the HFD and were treated with RRx-001 (HFD-RRx-001). The HDL performs a role of removing other forms of cholesterol such as LDL from the bloodstream.

As shown in FIG. 1B, the ND rats (e.g., the first group) had an amount of HDL that was normalized to 1. The amount of HDL for the HFD rats (e.g., the second group) was approximately 0.2 relative to the ND rats (e.g., the first group). The amount of the HDL in the HFD-RRx-001 rats (e.g., the third group) was approximately 0.4 relative to the ND rats (e.g., the first group). Accordingly, the treatment of RRx-001 correlated to an increase in the HDL in rats that were fed a HFD.

FIG. 1C depicts a bar graph 140 of a relative amount of triglycerides (TG) in the first group of the rats that were fed the ND, the second group of the rats that were fed the HFD, and the third group of the rats that were fed the HFD and were treated with RRx-001 (HFD-RRx-001). TGs are a common form of fat that is stored in the body. High levels of TGs are associated with various negative health conditions, such as obesity and diabetes.

As shown in FIG. 1C, the ND rats has an amount of TG that was normalized to 1. The amount of TG for the HFD rats (e.g., the second group) was approximately 6.2 relative to the ND rats (e.g., the first group). The amount of HDL in the HFD-RRx-001 rats (e.g., the third group) was approximately 4.4 relative to the ND rats (e.g., the first group).

FIG. 1D depicts a bar graph 160 of a relative amount of total cholesterol (TC) in the first group of the rats that were fed the ND, the second group of the rats that were fed the HFD, and the third group of the rats that were fed the HFD and were treated with RRx-001 (HFD-RRx-001). The TC is a measure of multiple lipids in the blood, including LDL, HDL, and triglycerides.

As shown in FIG. 1D, the ND rats (e.g., the first group) had an amount of the TC that was normalized to 1. The TC for the HFD rats (e.g., the second group) was approximately 2.7 times relative to the ND rats (e.g., the first group). The TC of the HFD-RRx-001 rats (e.g., the third group) was approximately 1.8 times relative to the ND rats (e.g., the first group).

Accordingly, as shown in FIG. 1A-FIG. 1D, administration of RRx-001 in HFD rats (e.g., the third group) is correlated to lipid levels of LDL, HDL, triglycerides, and total cholesterol that is closer to rats with the ND (e.g., the first group) than with the HFD alone (e.g., the second group).

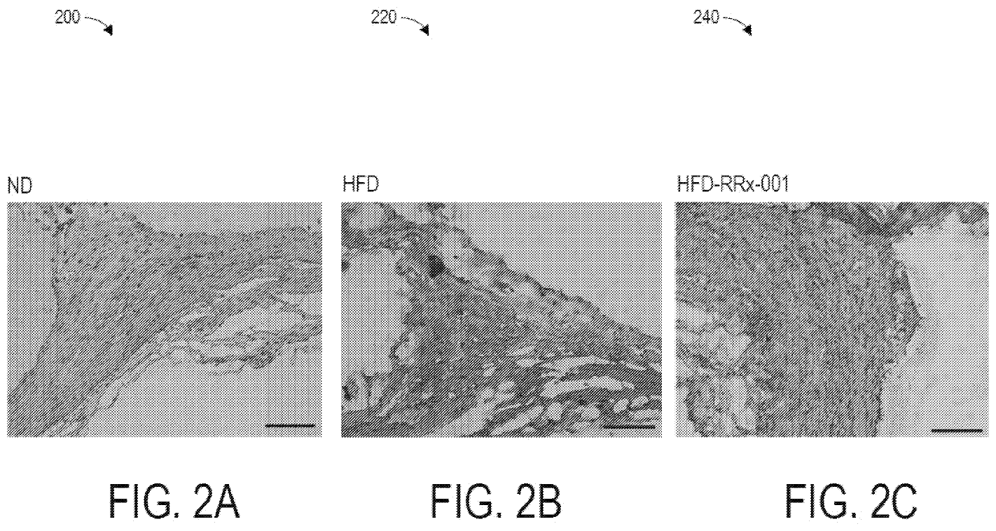

Oil red O stains lipids including atherosclerotic plaques. Accordingly, plaque deposits in arteries may be visualized via oil red O staining. As shown in FIG. 2A, which depicts an oil red O staining 200 of aortic tissues of a rat that was fed the ND (e.g., the first group), the aortic tissue of the ND rat shows minimal amounts of oil red O staining. Thus, the ND rats (e.g., the first group) had little to no plaque deposits in its artery. As shown in FIG. 2B, which depicts an oil red O staining 220 of aortic tissues of a rat that was fed the HFD (e.g., the second group), the aortic tissue of the HFD rat had substantial staining, indicating lipids and significant build-up of plaque in its arteries. As shown in FIG. 2C, which depicts an oil red O staining 240 of aortic tissues of a rat that was fed the HFD-RRx-001 (e.g., the third group), the aortic tissue had a color that was approximately halfway in between the significant staining the HFD tissue (e.g., FIG. 2B) and the lack of staining for the ND tissue (e.g., FIG. 2A). As such, administration of a therapeutically effective dose of RRx-001 mitigates build-up of plaque in high fat conditions.

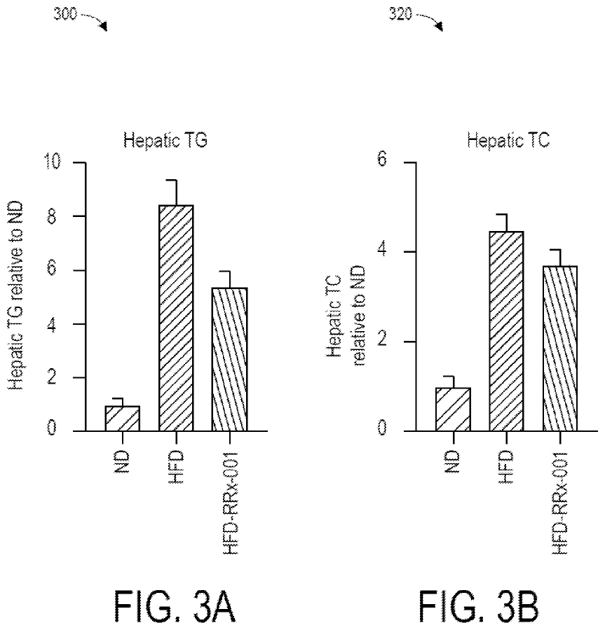

FIG. 3A is a bar graph 300 of a relative amount of hepatic TGs in the first group of the rats that were fed the ND, the second group of the rats that were fed the HFD, and the third group of the rats that were fed the HFD and were treated with RRx-001 (HFD-RRx-001). Hepatic TGs are TGs found in a sample of the liver and are strongly associated with NASH. The ND rats (e.g., the first group) had hepatic TG amounts normalized to 1. The HFD rats (e.g., the second group) had amounts of hepatic TGs at approximately 8.2 times relative to the amounts for the ND rats (e.g., the first group). The HFD-RRx-001 rats (e.g., the third group) had amounts of hepatic TGs at approximately 5.7 times relative to the amounts of the ND rats (e.g., the first group). Thus, administration of RRx-001 mitigates the hepatic TGs in rats that are fed the HFD.

FIG. 3B is a bar graph 320 of a relative amount of hepatic TC in the first group of the rats that were fed the ND, the second group of the rats that were fed the HFD, and the third group of the rats that were fed the HFD and were treated with RRx-001 (HFD-RRx-001). The hepatic TC is a measure of a multitude of lipids in liver tissue. A severity of diseases of the liver may be measured in part by a hepatic TC.

As shown in FIG. 3B, the hepatic TC amounts for rats that were fed the ND (e.g., the first group) was normalized to 1. The HFD rats (e.g., the second group) had hepatic TC amounts at approximately 4.3 times relative to the ND rats (e.g., the first group). The HFD-RRx-001 rats (e.g., the third group) had hepatic TC amounts at approximately 3.8 times relative to the ND rats (e.g., the first group). Similar to the other results presented herein, administering RRx-001 to HFD rats at a high risk of developing medical conditions associated with high fat mitigates or reduces the amount of hepatic TC.

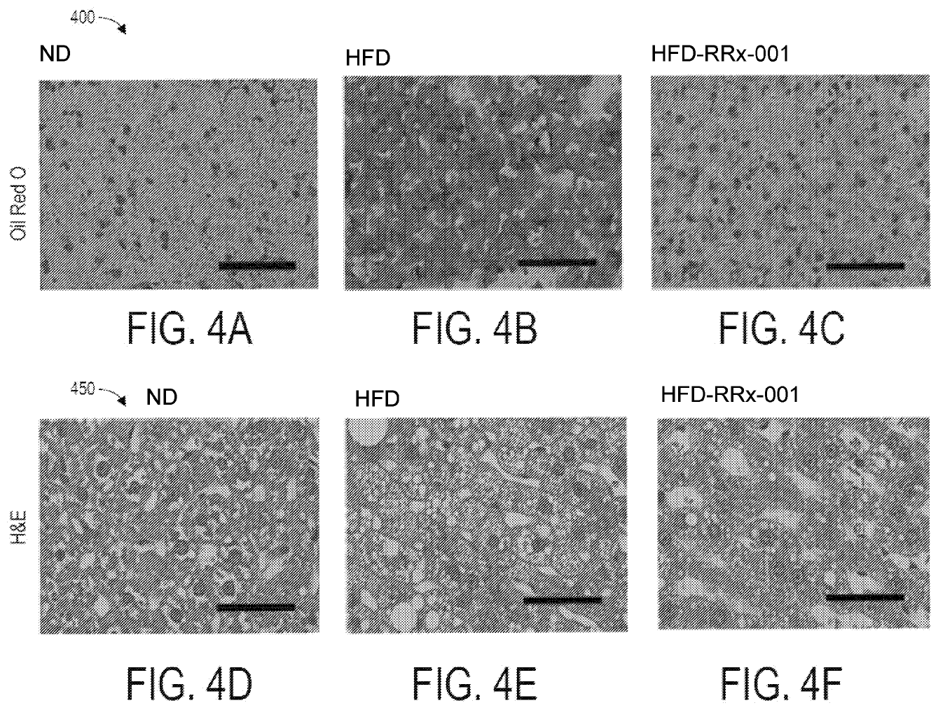

FIG. 4A, FIG. 4B, and FIG. 4C depict tissue samples 400 that were stained with oil red O for rats that were fed the ND, the HFD, and the HFD-RRx-001, respectively. As shown in FIG. 4A, the rats that were fed the ND (e.g., the first group) shows minimal staining. As shown in FIG. 4B, the HFD tissue (e.g., the second group of the rats) shows substantial staining, which indicates a high amount of lipids in the sample. FIG. 4C shows intermediate staining that is in between the substantial staining for the HFD (e.g., FIG. 4B) and the minimal staining for the ND (e.g., FIG. 4A). Accordingly, administration of RRx-001 mitigates the amount of lipids in tissues.

FIG. 4D, FIG. 4E, and FIG. 4F depict tissue samples 450 that are stained with H&E for rats that were the ND (e.g., the first group), the HFD (e.g., the second group), and the HFD-RRx-001 (e.g., the third group), respectively. The H&E stain colors different parts of a cell different colors, thus making the cell nucleus and cytoplasm easily distinguishable from one another. As shown in FIG. 4D, FIG. 4E, and FIG. 4F, the H&E stains for the rats that were fed the ND (e.g., the first group), the HFD (e.g., the second group), and the HFD-RRx-001 (e.g., the third group) appear similar.

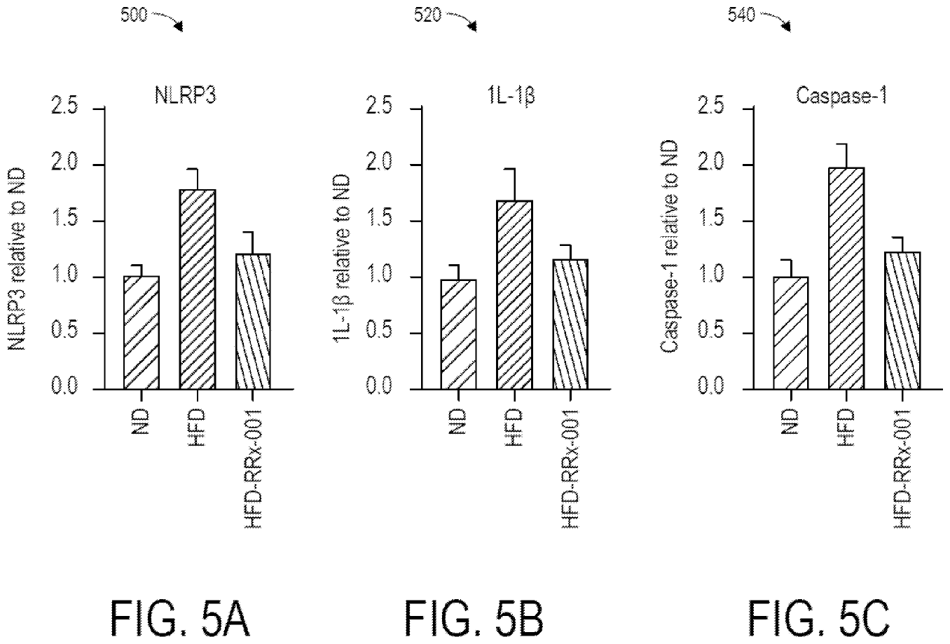

FIG. 5A depicts a bar graph 500 of a relative amount of the serum NLRP3 in the first group of the rats that were fed the ND, the second group of the rats that were fed the HFD, and the third group of the rats that were fed the HFD and were treated with RRx-001 (HFD-RRx-001). The NLRP3 is a sensor for intracellular danger signals and is a component of NLRP3 inflammasome. The NLRP3 inflammasome is made up of the NLRP3, adaptor protein ASC, and pro-caspase-1. The NLRP3 mediates production of the IL-1β.

As shown in FIG. 5A, the amount of the serum NLRP3 in rats that were fed the ND (e.g., the first group) was normalized to 1. The amount of serum NLRP3 in the rats that were fed the HFD (e.g., the second group) was approximately 1.75 times relative to the rats that were fed the ND (e.g., the first group). The amount of the serum NLRP3 in the rats that were fed the HFD-RRx-001 (e.g., the third group) had an amount of the serum NLRP3 at approximately 1.3 times relative to the rats that were fed the ND (e.g., the first group).

FIG. 5B depicts a bar graph 520 of a relative amount of IL-1β in rats that were fed the ND (e.g., the first group), the HFD (e.g., the second group), and the HFD-RRx-001 (e.g., the third group). The IL-1β is an inflammatory cytokine that has multiple functions and is mediated by the NLRP3. As shown in FIG. 5B, the amount of the IL-1β in the rats that were fed the ND (e.g., the first group) was normalized to 1. The amount of the IL-1β in the rats that were fed the HFD (e.g., the second group) was approximately 1.7 times relative to the amount of the IL-1β in the ND rats (e.g., the first group). The amount of the IL-1β in HFD-RRx-001 (e.g., the third group) was approximately 1.2 times relative to the amount in the ND rats (e.g., the first group).

FIG. 5C depicts a bar graph 540 of a relative amount of caspase-1 in the rats that were fed the ND (e.g., the first group), the HFD (e.g., the second group), and the HFD-RRx-001 (e.g., the third group). Caspase-1 converts that inactive form of the IL-1β to an active inflammatory cytokine. As shown in FIG. 5C, the amount of the caspase-1 in the rats that were fed the ND (e.g., the first group) was normalized to 1. The amount of caspase-1 in the rats that were fed the HFD (e.g., the second group) was approximately 2.0 times relative to the rats that were fed the ND (e.g., the first group). The amount of the caspase-1 in the rats that were fed the HFD-RRx-001 (e.g., the third group) was approximately 1.3 times relative to the rats that were fed the ND (e.g., the first group).

As shown in FIG. 1-FIG. 5C, RRx-001 reduced inflammation with an anti-lipidemic and anti-NASH effect mediated at least in part by inhibition of inflammatory pathways. Thus, targeting inflammatory pathways with RRx-001 contributes to the prevention and treatment of medical conditions that correlate to dyslipidemia, such as NASH and atherosclerosis.

Many variations may be made to the embodiments described herein. All variations and combinations of variations are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

### Other Embodiments

This application refers to various published patent applications, journal articles, and other publications, each of which is incorporated herein by reference.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein are incorporated by reference in their entirety for all purposes.

What is claimed is:

1. A method for preventing or treating a disease or a disorder associated with non-alcoholic fat accumulation in a liver of a subject, the non-alcoholic fat accumulation in the liver being associated with or without fibrosis, cirrhosis, and hepatocellular carcinoma, the method comprising administering an effective amount of RRx-001, or a pharmaceutically acceptable salt thereof, to the subject in need thereof.

2. The method of claim 1, wherein the subject is a mammal subject.

3. The method of claim 2, wherein the mammal subject is a human subject.

4. The method of any of claims 1-3, wherein the effective amount of RRx-001, or the pharmaceutically acceptable salt thereof, is a therapeutically effective amount of the RRx-001, or the pharmaceutically acceptable salt thereof.

5. The method of claim 4, wherein the therapeutically effective amount of the RRx-001, or the pharmaceutically acceptable salt thereof, is in a range of from about 0.1 mg to about 500.0 mg.

6. The method of claim 5, wherein the therapeutically effective amount of the RRx-001, or the pharmaceutically acceptable salt thereof, is in a range of from about 0.5 mg to about 200.0 mg.

7. The method of any of claims 1-6, wherein the mammal subject is a non-human subject.

8. The method of claim 7, wherein the effective amount of RRx-001, or the pharmaceutically acceptable salt thereof, is a therapeutically effective amount of the RRx-001, or the pharmaceutically acceptable salt thereof.

9. The method of claim 8, wherein the therapeutically effective amount of the RRx-001, or the pharmaceutically acceptable salt thereof, is in a range of from about 0.1 mg/kg to about 100.0 mg/kg.

10. The method of claim 9, wherein the therapeutically effective amount of the RRx-001, or the pharmaceutically acceptable salt thereof, is in a range of from about 1 mg/kg to about 50.0 mg/kg.

11. The method of any of claims 1-10,
wherein the disease or the disorder is associated with dyslipidemia, and
wherein the disease or the disorder is selected from the group consisting of: non-alcoholic fatty liver disease (NAFLD), non-alcoholic steatohepatitis (NASH), atherosclerosis, hyperlipidemia, hypercholesterolemia, steatosis, liver fibrosis, coronary heart disease, peripheral vascular disease, stroke, aortic aneurysm, glucose intolerance, and diabetes.

12. The method of any of claims 1-11, wherein administering the effective amount of RRx-001, or the pharmaceutically acceptable salt thereof, to the subject in need thereof reduces at least one factor of the disease or the disorder associated with the dyslipidemia in the subject.

13. The method of claim 12, wherein each factor of the at least one factor of the disease or the disorder associated with the dyslipidemia in the subject is selected from the group consisting of: a hepatic steatosis score, a serum alanine transaminase (ALT) level, a serum aspartate transferase (AST) level, a hepatic level of cholesterol, a hepatic level of triglycerides, insulin resistance, hyperinsulinemia, a serum NLRP3 level, a fibrosis marker, an oxidative stress marker in the subject, an inflammatory stress marker in the subject, and a marker associated with LDL.

14. The method of claim 13,
wherein the at least one factor of the disease or the disorder associated with the dyslipidemia in the subject comprises the serum NLRP3 level, and
wherein reduction of the serum NLRP3 level is associated with suppression of NLRP3 inflammasome activation.

15. The method of claim 13,
wherein the at least one factor of the disease or the disorder associated with the dyslipidemia in the subject comprises the inflammatory stress marker or the oxidative stress marker in the subject, and
wherein the inflammatory stress marker or the oxidative stress marker is selected from the group consisting of: Interleukin-1 (IL-1), Interleukin-1 beta (or IL-1p), Interleukin-6 (IL-6), Interleukin-8 (IL-8), Interleukin-18 (IL-18), caspase-1, interferon-γ (IFN-γ), Monocyte chemoattractant protein-1 (MCP-1), Cyclooxygenase-2 (COX-2), C-reactive protein (CRP), isoprostanes (IsoPs) and malondialdehyde (MDA), nuclear factor-kappa B (NF-kβ), prostaglandins, and Tumor necrosis factor α (TNFα).

16. The method of claim 13,
wherein the at least one factor of the disease or the disorder associated with the dyslipidemia in the subject comprises the marker associated with the LDL, and
wherein the marker associated with the LDL is selected from the group consisting of: serum amyloid A-LDL (SAA-LDL) and α1-antitrypsin-LDL (AT-LDL).

17. The method of any of claims 1-16, wherein administering the effective amount of RRx-001, or the pharmaceutically acceptable salt thereof, is performed via a single administration.

18. The method of any of claims 1-16, wherein administering the effective amount of RRx-001, or the pharmaceutically acceptable salt thereof, is performed via at least two administrations.

19. The method of claim 18, wherein administering the effective amount of RRx-001, or the pharmaceutically acceptable salt thereof, is performed at a frequency of at least once per day for a time period.

20. The method of claim 18, wherein administering the effective amount of RRx-001, or the pharmaceutically acceptable salt thereof, is performed at a frequency of at least once per week for a time period.

21. The method of claim 18, wherein administering the effective amount of RRx-001, or the pharmaceutically acceptable salt thereof, is performed at a frequency of at least once per month for a time period.

22. The method of any of claims 1-21, wherein administering the effective amount of RRx-001, or the pharmaceutically acceptable salt thereof, occurs via parenteral administration.

23. The method of claim 22, wherein administering the effective amount of RRx-001, or the pharmaceutically acceptable salt thereof, occurs via an intravenous administration, intraarterial hepatic administration, an oral administration, intraperitoneal administration, rectal administration, topical administration, vaginal administration, direct intralesional injection, a subcutaneous administration, or an intramuscular administration.

24. The method of claim 23, wherein administering the effective amount of RRx-001, or the pharmaceutically acceptable salt thereof, occurs via the intravenous administration.

25. The method of any of claims 1-24, wherein the effective amount of RRx-001, or the pharmaceutically acceptable salt thereof, is administered prior to, concurrently with, or subsequent administration of an agent.

26. The method of any of claims 1-24, wherein the effective amount of RRx-001, or the pharmaceutically acceptable salt thereof, is administered in a pharmaceutical composition comprising an agent.

27. The method of any of claims 1-24, wherein the effective amount of RRx-001, or the pharmaceutically acceptable salt thereof, is administered in a first pharmaceutical composition.

28. The method of claim 27, wherein an agent is administered in a second pharmaceutical composition prior to, concurrently with, or subsequent the administration of the first pharmaceutical composition.

29. The method of any of claims 25-28, wherein the agent comprises a NASH therapy.

30. The method of claim 29, wherein the NASH therapy is selected from the group consisting of: an anti-inflammatory agent, an anti-diabetic agent, an anti-fibrotic agent, an anti-steatiotic agent, a cholesterol/lipid modulating agent, and an anti-diabetic agent.

31. The method of claim 29, wherein the NASH therapy is selected from the group consisting of: a statin, a PCSK9 inhibitor, a small interfering RNA, a selective microsomal triglyceride transfer protein inhibitor, an apolipoprotein B antisense oligonucleotide, a monoclonal antibody against angiopoietin-like protein 3 (ANGPTL3), Vitamin E, metformin, aspirin, a peroxisome proliferator-activated receptor (PPAR) agonist, a thyroid hormone receptor beta (THR-β) agonist, insulin, a sulfonylurea, a farnesoid X receptor (FXR) agonist, a glucagon-like peptide 1 (GLP-1) antagonist, a fibric acid derivative, a bile acid sequestrant, nicotinic acid, a selective cholesterol absorption inhibitor, red rice yeast, an omega 3 fatty acid, a fatty acid ester, and an adenosine triphosphate-citrate lyase (ACL) inhibitor.

32. The method of any of claims 1-24, wherein the effective amount of RRx-001, or a pharmaceutically acceptable salt thereof, is administered in a pharmaceutical composition comprising a blood product.

33. The method of claim 32, wherein the blood product comprises erythrocyte cells.

34. The method of claim 33, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

35. The method of claim 32, wherein the blood product is a mixture of packed red blood cells.

36. The method of claim 32, wherein the blood product is whole blood.

37. The method of claim 36, wherein the whole blood is autologous whole blood.

38. The method of any of claims 32-37, wherein the pharmaceutical composition further comprises an agent.

39. The method of claim 38, wherein the agent comprises a NASH therapy.

40. The method of claim 39, wherein the NASH therapy is selected from the group consisting of: an anti-inflammatory agent, an anti-diabetic agent, an anti-fibrotic agent, an anti-steatiotic agent, a cholesterol/lipid modulating agent, and an anti-diabetic agent.

41. The method of claim 39, wherein the NASH therapy is selected from the group consisting of: a statin, a PCSK9 inhibitor, a small interfering RNA, a selective microsomal triglyceride transfer protein inhibitor, an apolipoprotein B antisense oligonucleotide, a monoclonal antibody against angiopoietin-like protein 3 (ANGPTL3), Vitamin E, metformin, aspirin, a peroxisome proliferator-activated receptor (PPAR) agonist, a thyroid hormone receptor beta (THR-β) agonist, insulin, a sulfonylurea, a farnesoid X receptor (FXR) agonist, a glucagon-like peptide 1 (GLP-1) antagonist, a fibric acid derivative, a bile acid sequestrant, nicotinic acid, a selective cholesterol absorption inhibitor, red rice yeast, an omega 3 fatty acid, a fatty acid ester, and an adenosine triphosphate-citrate lyase (ACL) inhibitor.

42. A pharmaceutical composition for treating a disease or a disorder associated with dyslipidemia in a subject, the pharmaceutical composition comprising an effective amount of RRx-001, or a pharmaceutically acceptable salt thereof.

43. The pharmaceutical composition of claim 42, wherein the subject is a mammal subject.

44. The pharmaceutical composition of claim 43, wherein the mammal subject is a human subject.

45. The pharmaceutical composition of any of claims 42-44, wherein the effective amount of RRx-001, or the pharmaceutically acceptable salt thereof, is a therapeutically effective amount of the RRx-001, or the pharmaceutically acceptable salt thereof.

46. The pharmaceutical composition of claim 45, wherein the therapeutically effective amount of the RRx-001, or the pharmaceutically acceptable salt thereof, is in a range of from about 0.1 mg to about 500.0 mg.

47. The pharmaceutical composition of claim 46, wherein the therapeutically effective amount of the RRx-001, or the pharmaceutically acceptable salt thereof, is in a range of from about 0.5 mg to about 200.0 mg.

48. The pharmaceutical composition of claim 42, wherein the mammal subject is a non-human subject.

49. The pharmaceutical composition of claim 48, wherein the effective amount of RRx-001, or the pharmaceutically acceptable salt thereof, is a therapeutically effective amount of the RRx-001, or the pharmaceutically acceptable salt thereof.

50. The pharmaceutical composition of claim 49, wherein the therapeutically effective amount of the RRx-001, or the pharmaceutically acceptable salt thereof, is in a range of from about 0.1 mg/kg to about 100.0 mg/kg.

51. The pharmaceutical composition of claim 50, wherein the therapeutically effective amount of the RRx-001, or the pharmaceutically acceptable salt thereof, is in a range of from about 1 mg/kg to about 50.0 mg/kg.

52. The pharmaceutical composition of any of claims 42-51,
wherein the disease or the disorder is associated with dyslipidemia, and
wherein the disease or the disorder is selected from the group consisting of: non-alcoholic fatty liver disease (NAFLD), non-alcoholic steatohepatitis (NASH), atherosclerosis, hyperlipidemia, hypercholesterolemia, steatosis, liver fibrosis, coronary heart disease, peripheral vascular disease, stroke, aortic aneurysm, glucose intolerance, and diabetes.

53. The pharmaceutical composition of any of claims 42-52, wherein the effective amount of RRx-001, or a pharmaceutically acceptable salt thereof reduces at least one factor of a disease or a disorder associated with dyslipidemia in the subject.

54. The pharmaceutical composition of claim 53, wherein each factor of the at least one factor of the disease or the disorder associated with the dyslipidemia in the subject is selected from the group consisting of: a hepatic steatosis score, a serum alanine transaminase (ALT) level, a serum aspartate transferase (AST) level, a hepatic level of cholesterol, a hepatic level of triglycerides, insulin resistance, hyperinsulinemia, a serum NLRP3 level, a fibrosis marker, an oxidative stress marker in the subject, an inflammatory stress marker in the subject, and a marker associated with LDL.

55. The pharmaceutical composition of claim 54,
wherein the at least one factor of the disease or the disorder associated with the dyslipidemia in the subject comprises the serum NLRP3 level, and
wherein reduction of the serum NLRP3 level is associated with suppression of NLRP3 inflammasome activation.

56. The pharmaceutical composition of claim 54,
wherein the at least one factor of the disease or the disorder associated with the dyslipidemia in the subject comprises the inflammatory stress marker in the subject, and
wherein the inflammatory stress marker or the oxidative stress marker is selected from the group consisting of: Interleukin-1 (IL-1), IL-1 beta (or IL-1p), Interleukin-6 (IL-6), Interleukin-8 (IL-8), Interleukin-18 (IL-18), caspase-1, interferon-γ (IFN-γ), Monocyte chemoattractant protein-1 (MCP-1), Cyclooxygenase-2 (COX-2), C-reactive protein (CRP), isoprostanes (IsoPs), malondialdehyde (MDA), nuclear factor-kappa B (NF-kβ), prostaglandins, and Tumor necrosis factor α (TNFα).

57. The pharmaceutical composition of claim 54,
wherein the at least one factor of the disease or the disorder associated with the dyslipidemia in the subject comprises the marker associated with the LDL, and
wherein the marker associated with the LDL is selected from the group consisting of: serum amyloid A-LDL (SAA-LDL) and α1-antitrypsin-LDL (AT-LDL).

58. The pharmaceutical composition of any of claims 42-57, wherein administering the effective amount of RRx-001, or the pharmaceutically acceptable salt thereof, is performed via a single administration.

59. The pharmaceutical composition of any of claims 42-57, wherein administering the effective amount of RRx-001, or the pharmaceutically acceptable salt thereof, is performed via at least two administrations.

60. The pharmaceutical composition of claim 59, wherein administering the effective amount of RRx-001, or the pharmaceutically acceptable salt thereof, is performed at a frequency of at least once per day for a time period.

61. The pharmaceutical composition of claim 59, wherein administering the effective amount of RRx-001, or the pharmaceutically acceptable salt thereof, is performed at a frequency of at least once per week for a time period.

62. The pharmaceutical composition of claim 59, wherein administering the effective amount of RRx-001, or the pharmaceutically acceptable salt thereof, is performed at a frequency of at least once per month for a time period.

63. The pharmaceutical composition of any of claims 42-62, wherein administering the effective amount of RRx-001, or the pharmaceutically acceptable salt thereof, occurs via parenteral administration.

64. The pharmaceutical composition of claim 63, wherein administering the effective amount of RRx-001, or the pharmaceutically acceptable salt thereof, occurs via an intravenous administration, intraarterial hepatic administration, an oral administration, intraperitoneal administration, rectal administration, topical administration, vaginal administration, direct intralesional injection, a subcutaneous administration, or an intramuscular administration.

65. The pharmaceutical composition of claim 64, wherein administering the effective amount of RRx-001, or the pharmaceutically acceptable salt thereof, occurs via an intravenous administration.

66. The pharmaceutical composition of any of claims 42-65, further comprising a blood product.

67. The pharmaceutical composition of claim 66, wherein the blood product comprises erythrocyte cells.

68. The pharmaceutical composition of claim 67, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

69. The pharmaceutical composition of claim 66, wherein the blood product is a mixture of packed red blood cells.

70. The pharmaceutical composition of claim 66, wherein the blood product is whole blood.

71. The pharmaceutical composition of claim 70, wherein the whole blood is autologous whole blood.

72. The pharmaceutical composition of any of claims 42-71, further comprising an agent.

73. The pharmaceutical composition of claim 72, wherein the agent comprises a NASH therapy.

74. The pharmaceutical composition of claim 73, wherein the NASH therapy is selected from the group consisting of: an anti-inflammatory agent, an anti-diabetic agent, an anti-fibrotic agent, an anti-steatiotic agent, a cholesterol/lipid modulating agent, and an anti-diabetic agent.

75. The pharmaceutical composition of claim 73, wherein the NASH therapy is selected from the group consisting of: a statin, a PCSK9 inhibitor, a small interfering RNA, a selective microsomal triglyceride transfer protein inhibitor, an apolipoprotein B antisense oligonucleotide, a monoclonal antibody against angiopoietin-like protein 3 (ANGPTL3), Vitamin E, metformin, aspirin, a peroxisome proliferator-activated receptor (PPAR) agonist, a thyroid hormone receptor beta (THR-β) agonist, insulin, a sulfonylurea, a farnesoid X receptor (FXR) agonist, a glucagon-like peptide 1 (GLP-1) antagonist, a fibric acid derivative, a bile acid sequestrant, nicotinic acid, a selective cholesterol absorption inhibitor, red rice yeast, an omega 3 fatty acid, a fatty acid ester, and an adenosine triphosphate-citrate lyase (ACL) inhibitor.

76. The pharmaceutical composition of any of claims 42-75, wherein the pharmaceutical composition is formulated as an aerosol formulation.
